(19) United States
(12) Patent Application Publication
(10) Pub. No.: US 2015/0320408 A1
(43) Pub. Date: Nov. 12, 2015

(54) Balloon Catheter for Treating Enteroatmospheric Fistulae in an Open Abdomen

(71) Applicant: Georgios ADAMIDIS, Zweibrücken (DE)

(72) Inventor: Georgios Adamidis, Zweibrucken (DE)

(21) Appl. No.: 14/651,715

(22) PCT Filed: Nov. 12, 2013
(86) PCT No.: PCT/EP2013/003402

§ 371 Date: Jun. 12, 2015

(30) Foreign Application Priority Data
Dec. 12, 2012 (DE) 10 2012 024 254.9

(51) Int. Cl. A61B 17/00

(52) U.S. Cl. CPC A61B 17/0057; A61B 17/12045

(57) ABSTRACT

The invention relates to a balloon catheter for treating enteroatmospheric fistulae in an open abdomen, consisting of an insertion tube that has a distal balloon arranged around the shaft and a proximal balloon arranged beneath this for sealing off the fistula, at least one injection line for filling said distal and proximal balloons with a filling medium, as well as a passage tube which is arranged transversely to said insertion tube at its base end and the lumen of which is connected to the lumen of the insertion tube and may be inserted into the intestinal tract.

### TECHNICAL AREA

The present invention relates to a balloon catheter for treating enteroatmospheric fistulae in an open abdomen.

### PRIOR ART

Enterocutaneous fistulae are produced as a consequence of inflammatory intestinal diseases (e.g. Colitis ulcerosa, Morbus Crohn) or as a complication in surgical interventions in the gastrointestinal tract. Enterocutaneous fistulae are a non-natural connection path between the intestine and the skin of the abdominal wall. In the case of such an open abdomen the mortality of the persons concerned rises by the formation of an intestinal fistula to 40 to 60% (Schein M S, Decker G A. Postoperative external alimentary tract fistulas. Am J Sug. 1191; 161: 435-438).

Intestinal fistulae frequently cause a peritonitis that drastically elevates the severity of the disease. The attempt has been made for some time with the aid of enterostomy to conduct the discharges of the intestine through the abdominal wall via a surgically produced opening of a part of the intestine. Discharge paths are possible in different intestinal parts, for example, in the ileum, caecum, sigmoid colon or transverse colon.

In contrast to enterocutaneous fistulae, fistulae that terminate in the exposed area of the open abdomen are designated as “enteroatmospheric”. The previously known removal systems, adhesive and sewing techniques as well as defect coverings are not a reliable treatment option. The patents frequently die from the complications. The morbidity and mortality in the treatment of an open abdomen was improved with the introduction of vacuum therapy (van Hensbroek P B, Wind J, Dijkgraaf M G W et al: Temporary closure of the open abdomen; A systematic review on delayed primary fascial closure in patients with an open abdomen. World J Surg. 2009; 33: 199-2070 Wild T, Stortecky S, Stremitzer S et al: Abdominal Dressing—A New Standard in the Treatment of the Open Abdomen As a Consequence of Secondary Peritonitis. Zentralabl Chir. 2006; 131: 111-114). However, vacuum therapy has the disadvantage that the fistula and therefore also the perforation of the intestinal wall are frequently enlarged on account of the active vacuum. This has the results that additional discharges pass to the outside. Additionally, a blockage of the suction sponge used in the vacuum therapy occurs on account of the exiting, often viscous secretion which reduces the effectiveness of the suction. Contaminations, infections and problems with wound healing threaten on account of the exiting discharges. Therefore, vacuum therapy is not a satisfactory treatment method.

Attempts have been made to separate the fistula from the vacuum system and to prevent an adhesion of the sponge but such measures are clearly more complicated (Goverman J, Yelon J A, Platz J J et al. The “Fistula VAC”, a technique for management of enterocutanous fistulae arising within the open abdomen: Report of 5 cases. J Trauma, 2006; 60: 428-431/Brunner W. Walzel G. Modifiziertes V.A.C.—Modified V.A.C. System in the Treatment of Enteral Fistulae in an Open Abdomen: Innovation—Indication-Technical Bases, ZfW., 2009: No. A: 56-58).

An alternative solution provides the using of a fistula adapter consisting of a cylinder of flexible material in order to minimize the mechanical irritation of the viscera (Jannasch Olof, Hans Lippert, Jörg Tautenhahn: “ A New Adapter for Supplying Enteroatmospheric Fistulae in an Open Abdomen”; Pharmetra, 2010). The adapter is inserted into an appropriately cut opening in the polyurethane sponge. Subsequently, the sponge including the fistula adapter is placed onto the wound. The PU sponge can be completely adhered over with a polyethylene sheet. Subsequently, a bipartite stoma set is adhered on. The fistula adapter is reliably held in the selected position by the vacuum present. The problems of vacuum therapy are reduced when using a fistula adapter; however, even a fistula adapter is not a reliable method in the treatment of enteroatmospheric fistulae.

In addition, there were attempts to use nourishment catheters or urine catheters to treat enteroatmospheric fistulae (Everson A R, Fischer J E.; Current management of enterocutaneous fistula. J Gastrointest Surg. 2006; 10: 455-464/Medeiros A C, Aires-Neto T, Marchini J S et al. Treatment of postoperative enterocutaneous fistulas by high-pressure vacuum with normal oral diet. Dig Surg. 2004; 21: 401-405). However, such catheters fail to operate on account of the frequently viscous secretion, which regularly caused blockages of the catheter lumen. Even an increase in size of the fistulae was frequently able to be observed in the case of blocked catheters.

Another device for removing secretions provides a suction attachment that is applied with its bottom on the fistula (Layton B, DuBose J. Nichols S et al. Pacifiying the open abdomen with concomitant intestinal fistula; a novel approach. Am J Surg. 2010; 199: e48-e50).

Therefore, based on the special problems in the treatment of enteroatmospheric fistulae and on the requirements in the removal of viscous secretion through the abdominal wall, the previously known catheter systems like those described, for example in DE 60 2005 005 567 T2, DE 11 2006 002 272 T5 or DE 11 2008 003 106 T5 are not suitable.

DE 10 2010 019 795 A1 describes a double balloon catheter system that should be used to seal puncture sites or openings in body cavities, hollow organs or in the case of percutaneous drainages in mammals. However, in the treatment of enteroatmospheric fistulae such a balloon catheter system would not be suitable since the two balloons would exert too much pressure on the intestinal wall, which would result in necroses in the fistula region. EP 1 022 033 A1 describes a catheter for the intravascular connection of two vessel sections that, however, is not fixed with balloons and in which, in addition, the line to the fluid supply as well as the connection line through which vascular fluid can flow have different volumes.

### PRESENTATION OF THE INVENTION

Given this background, the present invention has the problem of making an improved or alternative catheter available that is suitable for the effective treatment of enteroatmospheric fistulae in an open abdomen.

This problem is solved by a balloon catheter with the features of claim 1.

Preferred embodiments are found in the subclaims.

The double balloon catheter in accordance with the invention consists of an introductory tube hollow on the inside with a preferably approximately cylindrical cross section. The introductory tube has a lumen for the removal of intestinal secretions and for the introduction of washing liquid or medicaments into the intestine. A first, distal balloon is arranged on the introductory tube and completely surrounds the shaft of the introductory tube. A second, proximal balloon is arranged underneath it. Both balloons serve to seal the fistula opening.

The distal balloon and the proximal balloon are filled via at least one injection line with a filling medium (e.g., air or NaCl solution) which causes the balloon skin to be inflated. A passage tube arranged transversely, i.e., at a right angle to the introductory tube is constructed at the foot end of the introductory tube, the lumen of which passage tube is connected to the lumen of the introductory tube and which can be introduced into the intestinal section in the fistula region. The introductory tube and the passage tube preferably form a T-piece. The T-piece is preferably a single structural component; however, the introductory tube and the passage tube can also be separate units that are assembled together as needed. The two balloons are preferably permanently connected to the introductory tube.

The passage tube preferably consists of an elastic, flexible material so that it can be readily introduced by the operator via the fistula opening into the interior of the intestinal section without unnecessarily widening the fistula opening. This procedure takes place in an extremely protective manner with the catheter of the invention. To this end, at first one end of the passage tube is guided via the fistula opening into the intestinal canal and subsequently the contralateral end of the passage tube is introduced into the intestinal canal in the opposite direction. The introductory tube is guided to the outside via the fistula opening and the open abdomen.

The fistula region is the area of the body of the afflicted person in which the open abdomen (i.e., the open abdominal wall) and the intestinal perforation are located. The fistula opening designates the penetration of the intestine open to the outside and, if required, the tissue located above it.

The intestinal contents and the secretions at the fistula region, in particular at the fistula opening are guided past through the passage tube located in the intestinal canal. The customarily occurring complications as they were described above are clearly reduced or even entirely avoided. In a preferred embodiment the passage tube of the balloon catheter system has an outside diameter that corresponds approximately to the inside diameter of the intestinal section in the fistula region. An additional fixing of the passage tube in the intestinal canal is possible, for example with an additional balloon that surrounds the passage tube. Such an embodiment is described further below.

After the catheter has been placed the distal and the proximal balloon are inflated via at least one injection line with filling medium, as a result of which the balloon volume increases. In a preferred embodiment each balloon has its own injection line, as a result of which the individual balloon volumes can be individually adjusted. In the state of use the distal balloon seals the fistula opening from the outside whereas the proximal balloon seals the perforation from the inside. As a result of the counteracting contact pressure of the two balloons the intestinal perforation or the fistula opening is effectively sealed. Preferably one or more suction platelets (for example, a sponge) are present between the two balloons in order to additionally seal off the fistula, receive moisture and dampen the pressure of both balloons at the fistula opening.

There is the danger, depending on the structural shape of the balloons that the balloons are pressed too strongly into the fistula opening and therefore unnecessarily widen them. In order to avoid this, the distal balloon and/or the proximal balloon are preferably designed in a club shape. As a consequence, the contact pressures act in a radius around the fistula opening at the club ends but not at the fistula opening itself. In addition, a liquid-tight seal is ensured. The pressure is shifted from the actual fistula opening into the edge area of the fistula region.

In a preferred embodiment the two balloons can move along the introductory tube. In an alternative embodiment the balloons are firmly connected to the introductory tube. The movable embodiment has the advantage that the two balloons can be adapted in accordance with the wall thickness of the intestinal canal, the diameter of the fistula opening and the surrounding tissue. In addition, an adaptation can take place via the filling volume of the individual balloons.

The volume of the distal balloon is preferably greater than the volume of the proximal balloon in order to effectively counteract the pressure of the intestinal passage.

In an alternative embodiment at least one additional inflatable balloon is arranged on the passage tube of the balloon catheter of the invention in order to fix the passage tube in the intestinal canal. Preferably, a balloon is present at both ends of the passage tube. This ensures a secure fixing of the system inside the intestinal canal. The intestinal contents are conducted through the passage tube past the fistula opening.

The balloon catheter in accordance with the invention can be used to manufacture a medical product for the treatment of enteroatmospheric fistulae in an open abdomen. The medical product consists, for example, of the balloon catheter in accordance with the invention, instructions for use and optionally an injector for filling the balloons via the injection lines with filling medium. Furthermore, replacement suction platelets can be added to the medical product.

### SHORT DESCRIPTION OF THE DRAWINGS

The invention is explained in detail in the following three drawings.

In the drawings

FIG. 1 shows an embodiment of the balloon catheter in accordance with the invention,

FIG. 2 shows an example for using the balloon catheter in accordance with the invention in the treatment of enteroatmospheric fistulae,

FIG. 3 shows another embodiment of the balloon catheter in accordance with the invention with additional balloons on the passage tube.

### WAYS OF CARRYING OUT THE INVENTION AND INDUSTRIAL APPLICABILITY

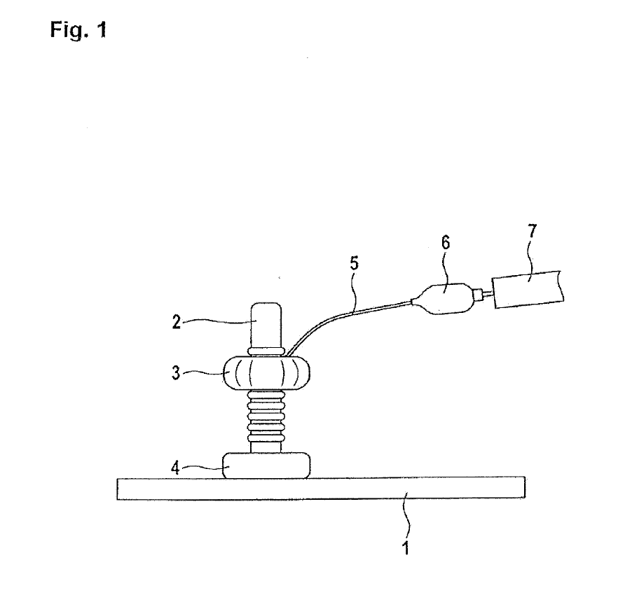

FIG. 1 shows the basic construction of the balloon catheter in accordance with the invention. An introductory tube 2 is connected to a passage tube 1 arranged transversely to it. The introductory tube 2 and the passage tube 1 form a T-piece. A distal balloon 3 is arranged around the shaft of the introductory tube 2. Underneath it a proximal balloon 4 is arranged that is also constructed on the shaft. In order to inflate the two balloons, 3, 4 at least one injection line 5 is provided. In order to fill the balloons 3, 4, for example, a solution of common salt or air can be used. The filling medium is preferably supplied via an injector 7. A valve 6 prevents a back flow of the injection liquid.

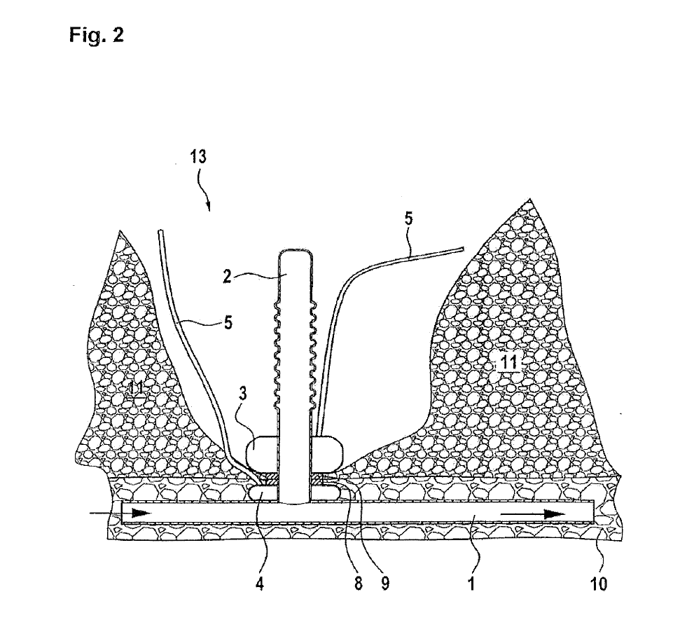

FIG. 2 shows an example of using the balloon catheter of the embodiment according to FIG. 1. It illustrates an open abdomen 13 with surrounding tissue 11 and with an intestinal canal 10. At first, a lateral end of the passage tube 1 is introduced through the fistula opening into the intestinal canal 10. Then, the contralateral end of the passage tube 1 is introduced in the opposite direction through the fistula opening. The proximal balloon 4 now sits in the intestinal lumen and presses against the inner wall whereas the distal balloon 3 presses from the outside against the tissue and the intestinal wall. Both balloons 3, 4 are filled with filling medium (air) via the separate injection lines 5. Both balloons 3, 4 are preferably shaped like a club so that the contact pressure in the fistula region is distributed in a greater radius around the fistula opening, which reduces or prevents a widening of the fistula opening. An upper suction platelet 9 and a lower suction platelet 8 are located between the two balloons 3, 4.

The intestinal contents can pass through the passage tube 1 without viscous secretion exiting via the introductory tube 2.

Washing liquid or medications can be introduced via the lumen of the introductory tube 2 (for example, from above) which, for example, prevents a clogging of the introductory tube 2 or of the passage tube 1 or makes a cleaning possible. For example, a solution of common salt can be used as washing liquid.

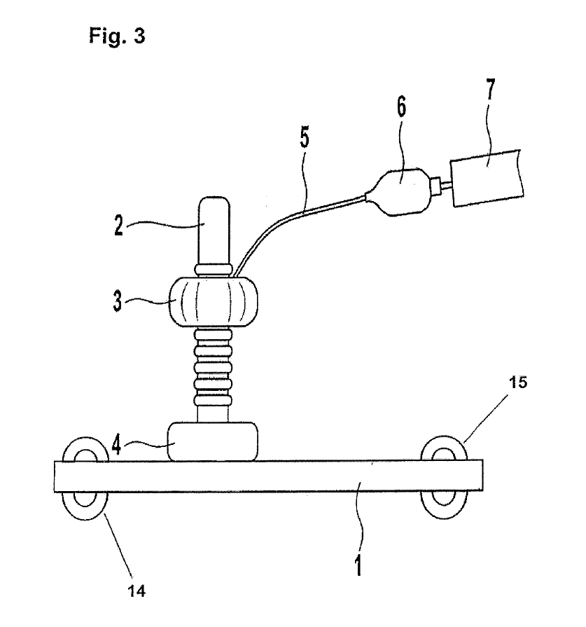

FIG. 3 shows another embodiment of the balloon catheter of the invention in which two additional balloons 14, 15 are arranged on the passage tube 1. The passage tube 1 consists of an elastic, flexible material, e.g. a PU plastic. The two balloons 14, 15 arranged on the end sides are filled with filling medium after the introduction of the passage tube 1 into the intestinal canal and inflated. This fixes the system in the intestinal canal. In as far as the balloons 14, 15 fill up the intestine in this region the intestinal contents are conducted through the passage tube 1.

The catheter in accordance with the invention is easy to manipulate, economical to manufacture and avoids the known complications.

1. A balloon catheter for treating enteroatmospheric fistulae in an open abdomen, comprising of an introductory tube with a distal balloon arranged around the shaft and a proximal balloon arranged underneath it for sealing off the fistulae, with at least one injection line for filling the distal and the proximal balloons with filling medium and with a passage tube arranged transversely to the introductory tube at its foot end and that the lumen of the passage tube is connected to the lumen of the introductory tube and can be introduced into the intestinal canal.

2. The balloon catheter according to claim 1, characterized in that the distal balloon and/or the proximal balloon is/are shaped like a club.

3. The balloon catheter according to claim 1, characterized in that the passage tube has an outside diameter that corresponds approximately to the inside diameter of the intestinal section in the fistula region.

4. The balloon catheter according to claim 1, characterized in that the passage tube consists of an elastic, flexible material.

5. The balloon catheter according to claim 1, characterized in that the introductory tube and the passage tube are constructed as a T-piece.

6. The balloon catheter according to claim 1, characterized in that one or more suction platelets are arranged between the distal balloon and the proximal balloon.

7. The balloon catheter according to claim 1, characterized in that the distal balloon has a greater volume in the inflated state than the proximal balloon.

8. The balloon catheter according to claim 1, characterized in that the distal balloon and the proximal balloon are each connected to its own injection line 5.

9. The balloon catheter according to claim 1, characterized in that at least one additional balloon is arranged on the passage tube.

10. (canceled)

11. The balloon catheter according to claim 2, characterized in that the passage tube has an outside diameter that corresponds approximately to the inside diameter of the intestinal section in the fistula region.
